(19) United States
(12) Patent Application Publication
(10) Pub. No.: US 2006/0282001 A1
(43) Pub. Date: Dec. 14, 2006

(54) PHYSIOLOGIC SENSOR APPARATUS

(76) Inventors: MICHEL NOEL, Sherbrooke (CA); SYLVAIN DUMONT, Sherbrooke (CA)

Correspondence Address: GOUDREAU GAGE DUBUC, 800 PLACE VICTORIA, SUITE 3400, MONTREAL, QUEBEC, H4Z 1E9, CA

(21) Appl. No.: 11/423,016

(22) Filed: Jun. 8, 2006

Related U.S. Application Data
(60) Provisional application No. 60/688,716, filed on Jun. 9, 2005.

(51) Int. Cl. A61B 5/08; A61B 5/00; A61B 5/04

(52) U.S. Cl. 600/535; 128/903; 600/323; 600/509

(57) ABSTRACT

An apparatus for sensing at least one physiological parameter of a patient and transmitting data related to the sensed physiological parameter to an external device. The apparatus comprises a detecting portion comprising at least one sensor interconnected with a connector, an acquisition portion comprising a connector interface configured to receive the connector, electronics for controlling the at least one sensor via the connector interface, receiving data related to the at least one physiological parameter from the at least one sensor via the connector interface and processing the received data, and a wireless interface for transmitting the processed data to the external device, and a power source for supplying energy to the acquisition module.

### CROSS REFERENCE TO RELATED APPLICATIONS

The present application claims priority from U.S. Provisional Application No. 60/688,716 the entire contents of which is incorporated herein by reference.

### FIELD OF THE INVENTION

The present invention relates to a physiologic sensor apparatus. In particular, the present invention relates to an apparatus having a multipart part design: a first part housing the detector; a second part housing an acquisition unit comprising a transmitter or transceiver (transmitter/receiver) and a third part housing a power source and which may contain electronics for carrying out electronic power management and/or to perform some less noise critical functions usually done by the acquisition unit. Each part of the design can be individually disposable, reusable and/or rechargeable.

### BACKGROUND OF THE INVENTION

The prior art reveals a number of sensor devices which collect data related to one or more physiological parameters of a patient and transmit this collected data via a wireless interconnection to an external device. One drawback of such prior art devices is that the power sources are typically integrated with the data acquisition portion of the device which as a result is bulky and must be worn on a belt strapped around the patient's waste, wrist or arm. One other drawback is that the data acquisition portion of the device is typically interconnected with the physiological parameter detecting portion using an expensive shielded cable. These cables are also moderately stiff which gives rise to noise artefacts and the like being introduced into the detected signals. In these systems, the data acquisition portion is usually relatively far from the signal source (i.e. at the other end of the cable connected to the sensor) increasing thereby the system's susceptibility to noise.

Furthermore, these cables limit patient mobility and require that health care personal assistance when patient is transported from one location to another.

### SUMMARY OF THE INVENTION

In order to address the above and other drawbacks, there is disclosed an apparatus for sensing at least one physiological parameter of a patient and transmitting data related to the sensed physiological parameter to an external device. The apparatus comprises a detecting portion comprising at least one sensor interconnected with a connector, an acquisition portion comprising a connector interface configured to receive the connector, electronics for controlling the at least one sensor via the connector interface, receiving data related to the at least one physiological parameter from the at least one sensor via the connector interface and processing the received data, and a wireless interface for transmitting the processed data to the external device, and a power source for supplying energy to the acquisition module.

In one embodiment, the physiological parameter of the patient is Sp0₂ and the sensor comprises a pair of LED emitters and a photodetector for detecting light emitted by the emitters.

In another embodiment the physiological parameter of the patient is respiration, and the sensor or acquisition unit illustratively comprises a first oscillator circuit comprising an oscillator, a first inductive elastic band configured to encircle the chest of the patient and a first output frequency which varies with a change in length of the first inductive elastic band, and a second oscillator circuit comprising an oscillator, a second inductive elastic band configured to encircle the abdomen of the patient and a second output frequency which varies with a change in length of the second inductive elastic band.

In an additional embodiment, the physiological parameter of the patient is again respiration and the sensor comprises: a first piezoelectric respiratory band comprising a piezoelectric material imbedded in a first elastic band configured to encircle the chest of the patient and a second piezoelectric respiratory band comprising a piezoelectric material imbedded in a second elastic band configured to encircle the abdomen of the patient.

In still another embodiment, the physiological parameter of the patient is an Electrocardiogram (ECG) and the sensor comprises electrodes interconnected with the connector via a lead.

There is also disclosed an apparatus for sensing at least one physiological parameter of a patient and transmitting data related to the sensed physiological parameter to an external device. The apparatus comprises a detecting/acquisition portion comprising at least one sensor, electronics operationally connected to the sensor for controlling the at least one sensor, receiving data related to the at least one physiological parameter from the at least one sensor and processing the received data, and a wireless interface for transmitting the processed data to the external device, and a power source for supplying energy to the electronics and the wireless interface.

The disclosed physiologic sensor apparatus additionally aims at reducing operational cost of operation of healthcare institutions, notably in operating rooms, intensive care wards and general medical care wards. The adoption of this wireless technology reduces costs by:
Improving reliability of monitoring and reducing measurements errors by reducing movement artefacts;
positively impacting healthcare and medical staff work efficiency by reducing the number of times sensors need to be uninstalled and reinstalled while having to move patient from one room to another; and
providing interoperability with both legacy and future telemetry systems, including electronic health records filing, reducing time for retrieving recorded data from a central data base, and improving data access from remote locations.

### BRIEF DESCRIPTION OF THE DRAWINGS

FIG. 1 is a top perspective view of a physiologic sensor in accordance with an illustrative embodiment of the present invention mounted on a patient's finger/hand/arm;

FIG. 2 is a top plan view of a physiologic sensor in accordance with an illustrative embodiment of the present invention;

FIG. 3 is a schematic diagram detailing the acquisition/detector of a physiologic sensor in accordance with an illustrative embodiment of the present invention;

FIG. 4 is a block diagram detailing the acquisition/detector of a physiologic sensor in accordance with an alternative illustrative embodiment of the present invention;

FIG. 5 is a front plan view of a patient wearing a respiration acquisition/detector in accordance with an alternative illustrative embodiment of the present invention;

FIG. 6 is a schematic diagram of a detector in accordance with an alternative illustrative embodiment of the present invention; and

FIG. 7 is a front plan view of a patient wearing an ECG acquisition/detector in accordance with an alternative illustrative embodiment of the present invention.

### DETAILED DESCRIPTION OF THE ILLUSTRATIVE EMBODIMENTS

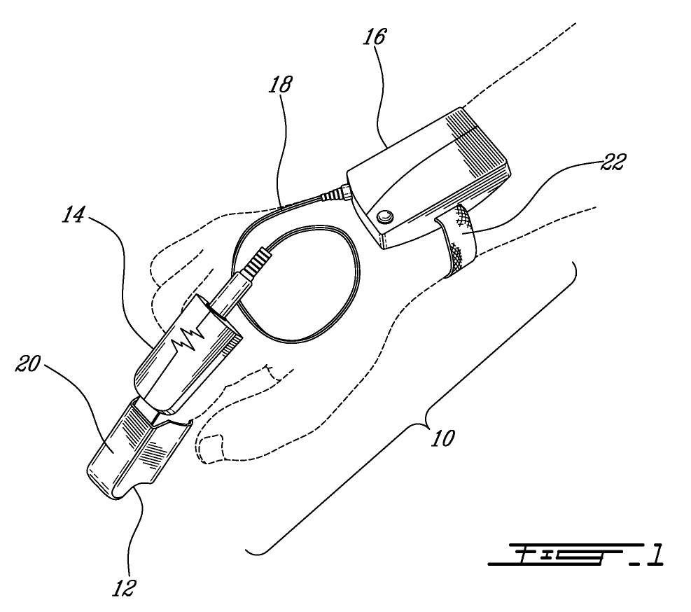

Referring now to FIG. 1, a physiologic sensor apparatus, generally referred to using the reference numeral 10, will now be described. The physiologic sensor apparatus 10 is comprised of a detector portion 12, an acquisition portion 14 and a power source 16. The power source 16, such as a replaceable battery or rechargeable battery or the like, supplies the acquisition portion 14 with power via an electrical wire 18. The detector portion 12, which can be either reusable or disposable, is secured to a patient through the use of, for example, an adhesive elastic bandage 20 which also provides support to the acquisition portion 14 attached to the detector portion 12 although in an alternative embodiment the acquisition portion 14 could be supported independently of the detector portion 12. Examples of suitable detector portions for use in this arrangement include those manufactured by Masimo and Nellcor, amongst others. Additionally, the power source 16 is mounted, for example, on a patient's wrist using an adjustable wrist strap 22. Note that “patient” as used herein should not be construed as being limited to humans, but may also include animals.

As will be discussed in more detail hereinbelow, the sensor apparatus 10 illustratively senses at least one physiological parameter of a patient and transmitting data related to the sensed physiological parameter to an external device.

Separating the detector portion 12 from the acquisition portion 14 (even if they are proximate to each other) provides for the use of a disposable detector portion 12 thereby reducing the risk of transmission of disease from patient to patient.

Additionally, maintaining the detector portion 12 and acquisition portion 14 proximate to one another increases the system's immunity to noise and also resolves the problem of cable length management usually required when connecting a detector portion 12 or sensor to a separate acquisition portion 14 using a specific length of shielded cable (as the required length may vary based on sensor type, patient condition, desired position and holding method).

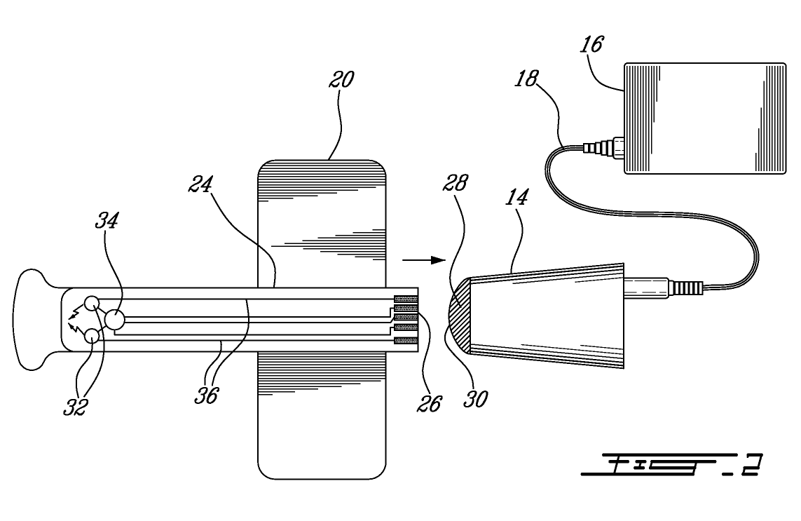

Referring now to FIG. 2 in addition to FIG. 1, illustratively, the detector portion 12 comprises sensing electronics and conductive traces mounted on a pliant substrate 24, for example a flexible printed circuit board (PCB), or the like. The substrate 24 also includes a connector 26. The connector 26 is inserted, for example, into a connector receiving slot 28 machined or otherwise formed in a forward end 30 of the acquisition portion 14. Given currently available battery sizes, removing the main power source 16 from encasement together with the electronics of the acquisition portion 14 provides for a significant reduction in the size of the acquisition portion 14. This in turn allows the acquisition portion 14 to be attached proximate to the detector portion 12 (which is typically mounted on a finger, toe, or the like) without reduction in the functions carried out by the acquisition portion 14. Such proximate attachment of the acquisition portion 14 to the detector portion 12 avoids the necessity of using an expensive shielded cable between the detector portion 12 and the acquisition portion 14. It also provides better immunity from noise, artefacts introduced into the detected signal associated with cable movement and resolves the problem of cable length management required when connecting a detector to a displaced acquisition module (as otherwise the required cable length may vary with the type of detector being used, the patient, desired position and method selected for securing the detector to the patient). Additionally, by using a very flexible wire for the electrical wire 18, which is possible as a shielded wire is no longer unnecessary, noise artefacts which would otherwise by introduced onto the detected signals by movement of the power source 16 are reduced.

By way of the acquisition portion 14 which is interconnected with the power source 16, power is supplied to the sensing electronics mounted on a pliant substrate 24.

Providing a power source 16 in a separate unit allows a lighter acquisition portion 14 to be provided. This approach is typically more comfortable for the patient and minimises motion artefacts arising, for example, from inertia of the heavier acquisition module. Also, when the power source 16 is provided in a separate unit, a different source of power can be selected to match the application while using the same sensor and acquisition portion 14.

Measurements related to at least one physiological parameter are sensed by the detector portion 12 and relayed to the acquisition portion 14. Illustratively, in order to collect SPO₂ measurements the detector portion 12 further comprises an emitter 32, comprised of one or more LEDs or the like, and a photodetector 34 in electrical contact with the connector 26 via a network of electrically conductive traces as in 36. As known in the art, light emitted by the LED emitters 32 is received by the photodetector 34 (typically via transmission of emitted light through a finger tip, toe or ear lobe, or reflection of the emitted light off a bone) which modulates the current flowing within the photodetector 34. The amount of light received by the photodetector 34, and therefore the current flowing through the photodetector 34, varies with the amount of oxygen in the patient's blood.

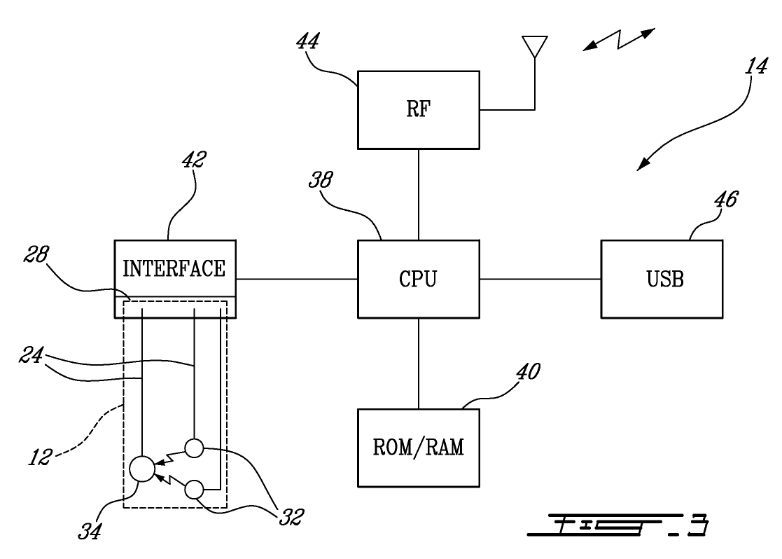

Referring to FIG. 3, in a first illustrative embodiment the acquisition portion 14 is microprocessor (or microcontroller) controlled and takes advantage of the sensing electronics of the detector portion 12 to collect measurements related to at least one physiological parameter, illustratively blood oximetry SPO2. The acquisition unit 14 is illustratively comprised of a CPU 38 which uses programs and parameters stored in a memory 40, illustratively ROM/RAM or flash memory, to control the collection of measurements related to the physiological parameters being collected. Additional devices peripheral to the CPU 36 comprise a sensor interface module 42, a wireless (RF) module 44 and optionally an external interface module 46, which may be a Universal Serial Bus (USB) or an other type (RS-232, Ethernet, infrared or other), which are included to allow the acquisition portion 14 to both collect data, transmit collected data to external devices (not shown) as well as receive configuration and other parameters from external programming devices (also not shown).

Still referring to FIG. 3, in order to collect measurements, the CPU 38 communicates with the interface module 42 which in turn controls the electronics of the detector 12 according to commands received from the CPU 38. As discussed above, in the illustrated example the electronics of the detector 12 comprise a pair of LED emitters 32 and a photodetector 34 which are interconnected with the interface module 42 via a series of electrically conductive traces 36 and the connector receiving slot 28 in the acquisition portion 14. The interface module 42 collects raw analog data from these electronics, whereby collected data is pre-processed by the interface module 42 prior to transfer to the CPU 38. Typical pre-processing includes, for example, amplifying and filtering of the analog signal and converting the analog signal into digital data.

Still referring to FIG. 3, in order to further process the digitized data the CPU 38 (or a Floating Point Unit, FPU) or the like as well as an onboard program allows, for example, an SPO₂ measurement algorithm to be applied to the digitized data. The CPU 38 also is illustratively able to store the results of such further processing in the memory 40 for subsequent retrieval and additional processing as required. Furthermore, the CPU 38 may transfer data to other external devices (not shown) via the wireless RF interface 44 or, optionally, provide for downloading of the data to such external devices via the external bus interface module 46, which, as stated above, may be an Universal Serial Bus (USB) or an other type (RS-232, Ethernet, infrared or the like).

Alternatively, the acquisition portion 14 could simply transmit a digitised version of the stream of raw analog data to an external device (not shown) which could subsequently carry out the processing to minimise power consumption and dimension of the acquisition unit.

The degree of post processing carried out may vary between specific applications. For example, increased post processing may be carried out where it is wished to reduce the RF bandwidth in hostile electromagnetic interference environments or less intensive (representation of the digitized analog raw data to an external device for further processing) in order to minimize power consumption and space.

Still referring to FIG. 3, interface (42) includes signal conditioning and an analog to digital converter. However, the analog to digital converter and RAM/ROM (40) can be integrated into the CPU (38), saving more space. The acquisition module may be based on discrete electronics (commercially available) or on a custom design to further decrease dimension.

The CPU 38 may transfer data to other external devices (not shown) via the wireless RF interface 44. In some specific applications, the CPU 38 may store the values (data) acquired by the acquisition portion 14 into the memory unit 40, for a determined period (number of hours), then CPU 38 may download the stored values (data) to other external devices (not shown) via the External Bus Interface 46 (USB or other).

Note that although the above illustrative embodiment of the present invention discloses a physiologic sensor apparatus 10 comprised of a separate detector portion 12, acquisition portion 14 and power source 16, it is within the scope of the present invention to integrate the detector portion 12, acquisition portion 14 and power source 16 into a single unit, or alternatively, to integrate the acquisition portion 14 together with the power source 16 or the detector portion 12 together with the acquisition portion 14.

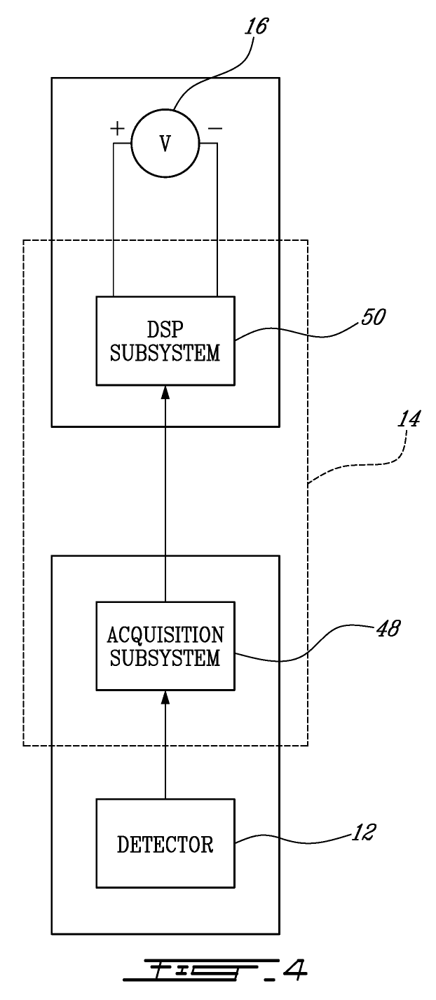

Similarly, and referring to FIG. 4, in an alternative embodiment the acquisition portion 14 may be divided into an analog acquisition subsystem 48 and a digital signal processing subsystem 50. In this regard, the analog acquisition subsystem 48 would typically comprise filters and other elements (not shown) to condition the analog signals and the digital signal processing subsystem 50 would comprise an A/D converter, digital processing unit and the RF transmitter or transceiver (also not shown). This would allow, for example and as illustrated, the analog acquisition subsystem 48 to be integrated in the same unit together with the detector portion 12 and the digital signal processing subsystem 50 to be integrated in the same unit together with the power source 16. This will typically simplify miniaturisation of the detector portion 12 and acquisition portion 14 (through, for example, the use of less electronics) which will in turn decrease movement artefacts while increasing patient comfort.

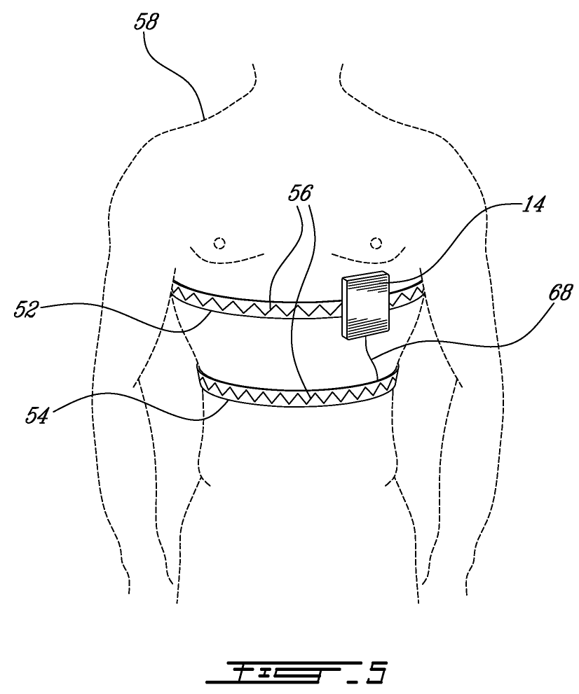

Referring now to FIG. 5, in an alternative embodiment of the present invention the separate detector portion can be a Respiratory Inductive Plethysmograph or RIP. As known in the art, an inductive plethysmograph is a non-invasive method to monitor patient ventilation by measuring the variation in the cross sectional area of the chest and the abdomen. Typically, first and second inductive elastic bands 52, 54 each illustratively comprising an inductive coil 56, for example a wire sewn in the elastic band in a zig zag fashion, which encircles the chest and the abdomen of the patient 58.

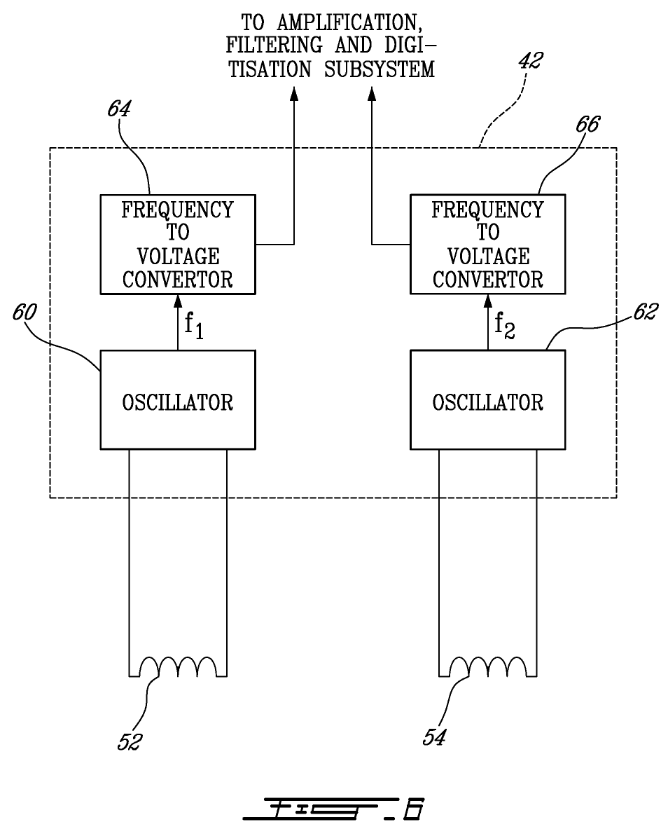

Referring now to FIG. 6, illustratively each inductive band 52, 54 forms an inductive element in a LC tank circuit (not shown), which determines the resonant frequency of a pair of oscillator circuits 60, 62 (illustratively Colpitt's oscillators) located in the interface 42 of the acquisition module 14. By measuring the oscillator frequency, changes in cross sectional area that occur in the elastic bands 52, 54 during breathing can be measured. Illustratively, a pair of frequency to voltage converters 64, 66 are provided to track the frequency change and produce a signal proportional to the oscillator frequency f₁, f₂ that can be amplified, filtered and digitized by amplification, filtering and digitising subsystems (all not shown).

Referring back to FIG. 5, as discussed above, each of the inductive bands 52, 54 encircle the chest and abdomen of a patient 58. Each band 52, 54 may have its own acquisition module 14 secured to it. Alternatively, and as illustrated in FIG. 4, a single acquisition module 14 may be shared by both bands 52, 54 and secured on one of the bands 52 or 54. In this case, a cable 68 is used to interconnect the second band 54 to the acquisition module 14. A connector interface such as the one illustrated in FIG. 5 is used to connect the acquisition module to the wire sewn in the elastic band.

In an alternative embodiment, the inductive plethysmograph could be replaced with a piezoelectric version of the same. In this regard, the manner of functioning of the piezoelectric based plethysmograph is analogous to that of the inductive plethysmograph. However, the pair of inductive bands 52, 54 are replaced with a pair of piezoelectric film-based respiratory bands. As known in the art, when piezoelectric materials are subject to mechanical forces a measurable electrical potential arises within the material. As inhalation and exhalation give rise to mechanical forces being exerted on the piezoelectric film-based respiratory bands, and therefore a measurable potential, this measurable potential may serve as input to the amplification, filtering and digitising subsystems without the necessity of first providing an oscillator circuit and a frequency to voltage converter.

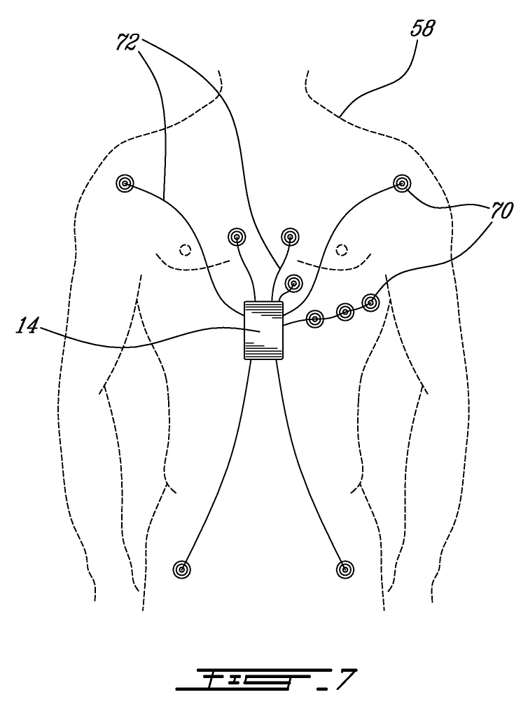

Referring now to FIG. 7, in still another alternative embodiment of the present invention the separate detector portion 12 can be an Electrocardiogram or ECG. As known in the art, the detecting portion of the ECG comprises a series of electrodes as in 70 which are attached to the patient 58 at certain predetermined locations on the patient 58. For example, in a 12 lead configuration six (6) electrodes as in 70 are placed on the chest and the remaining electrodes placed at locations based on Einthoven's triangle, such as the shoulders and thighs (as shown) or wrists and ankles (not shown). The electrodes as in 70 are in turn connected to the acquisition module 14 via a series of leads 72. Note that although a ten (10) electrode, twelve (12) lead placement is shown in FIG. 7, other configurations, such as 3 lead or 5 lead are known in the art and within the scope of the present. For example, a 3-lead standard electrode position is right arm, left arm and left leg. It is foreseen in the context of the present invention to use standard off-the-shelf electrodes for all of the above. The acquisition module 14 may be strapped to the arm, chest or any other patient location or placed into a pocket.

A connector interface (not shown) is provided for connecting the leads as in 72 to the acquisition module 14. Alternatively, the ends of leads as in 72 from a plurality electrodes as in 70 may be grouped to form a single cable which is plugged into the connector interface of the acquisition module 14. Alternatively, the leads as in 72 from the electrodes as in 70 may be replaced by conductive traces routed on a flexible substrate (for example a flexible printed circuit board, not shown) that connect to the acquisition module 14. The flexible substrate may also act as a holder for the acquisition module 14.

The acquisition module 14 illustratively processes the electrode potential from the electrical activity of the heart collected by the electrodes as in 70 and may carry out a number of signal conditioning operations, for example amplification, filtering and digitizing of the signals collected via the electrodes as in 70 and delivered to the acquisition module 14 via the leads as in 72.

Although the present invention has been described hereinabove by way of illustrative embodiments thereof, these embodiments can be modified at will without departing from the spirit and nature of the subject invention.

What is claimed is:

1. An apparatus for sensing at least one physiological parameter of a patient and transmitting data related to the sensed physiological parameter to an external device, the apparatus comprising:
a detecting portion comprising at least one sensor interconnected with a connector;
an acquisition portion comprising:
a connector interface configured to receive said connector;
electronics for controlling said at least one sensor via said connector interface, receiving output related to the at least one physiological parameter from said at least one sensor via said connector interface and processing said received output into data; and
a wireless interface for transmitting said processed data to the external device; and
a power source for supplying energy to said acquisition module.

2. The apparatus of claim 1, wherein acquisition portion comprises an analog acquisition subsystem and a digital signal processing subsystem and further wherein said analog acquisition subsystem and said detector portion are within a first unit and said digital signal processing subsystem is within a second unit separate from said first unit.

3. The apparatus of claim 2, wherein said digital signal processing subsystem and said power source are within said second unit.

4. The apparatus of claim 1, wherein said power source is a battery.

5. The apparatus of claim 4, wherein said battery is rechargeable.

6. The apparatus of claim 4, wherein said battery is disposable.

7. The apparatus of claim 1, wherein said acquisition portion and said power source are within a single unit.

8. The apparatus of claim 7, wherein said detecting portion, said acquisition portion and said power source are within a single unit.

9. The apparatus of claim 1, wherein said detector is disposable.

10. The apparatus of claim 1, wherein said detector is reusable.

11. The apparatus of claim 1, wherein said connector and each of said at least one sensor are mounted on a flexible substrate and interconnected via at least one conductive trace on said flexible substrate.

12. The apparatus of claim 1, wherein the at least one physiological parameter of the patient is Sp0₂, wherein said sensor comprises a pair of LED emitters and a photodetector for detecting light emitted by said emitters, and further wherein said output comprises an amount of light received by said photodetector, said amount of light varying in response to a change in a blood oxygen of the patient.

13. The apparatus of claim 1, wherein the at least one physiological parameter of the patient is respiration, and further wherein said at least one sensor comprises:
a first oscillator circuit comprising an oscillator and a first inductive elastic band configured to encircle the chest of the patient; and
a second oscillator circuit comprising an oscillator, a second inductive elastic band configured to encircle the abdomen of the patient;
wherein said output comprises a first output frequency which varies with a change in length of said first inductive elastic band and a second output frequency which varies with a change in length of said second inductive elastic band.

14. The apparatus of claim 13, wherein each of said oscillators is a Colpitt's oscillator.

15. The apparatus of claim 1, wherein the at least one physiological parameter of the patient is respiration, and further wherein said at least one sensor comprises:
a first inductive elastic band configured to encircle the chest of the patient; and
a second inductive elastic band configured to encircle the abdomen of the patient;
wherein said output comprises a first inductance which varies with a change in length of said first inductive elastic band and a second inductance which varies with a change in length of said second inductive elastic band.

16. The apparatus of claim 1, wherein the at least one physiological parameter of the patient is respiration, and further wherein said at least one sensor comprises:
a first piezoelectric respiratory band comprising a piezoelectric material imbedded in a first elastic band configured to encircle the chest of the patient; and
a second piezoelectric respiratory band comprising a piezoelectric material imbedded in a second elastic band configured to encircle the abdomen of the patient;
wherein said output comprises a first output voltage which varies with a change in length of said first elastic band and a second output voltage which varies with a change in length of said second elastic band.

17. The apparatus of claim 1, wherein the patient has a heart, the at least one physiological parameter of the patient is an ECG, wherein said at least one sensor comprises at least three (3) electrodes interconnected with said connector via a lead and further wherein said output comprises a voltage for each of said electrodes which varies in response to a change in an electrical activity of the heart.

18. An apparatus for sensing at least one physiological parameter of a patient and transmitting output related to the sensed physiological parameter to an external device, the apparatus comprising:
a detecting/acquisition portion comprising:
at least one sensor;
electronics operationally connected to said sensor for controlling said at least one sensor, receiving output related to the at least one physiological parameter from said at least one sensor and processing said received output; and
a wireless interface for transmitting said processed output to the external device; and
a power source for supplying energy to said electronics and said wireless interface.

19. The apparatus of claim 18, wherein said detecting/acquisition portion are within a single unit and said power source is an external power source.

20. The apparatus of claim 18, wherein said detecting/acquisition portion and said power source are within a single unit.
